(19) United States
(12) Patent Application Publication
(10) Pub. No.: US 2024/0341599 A1
(43) Pub. Date: Oct. 17, 2024

(54) Wireless ECG Device and System

(71) Applicant: Maria Bauza, Fairview, NC (US)

(72) Inventor: Maria Bauza, Fairview, NC (US)

(21) Appl. No.: 18/619,493

(22) Filed: Mar. 28, 2024

Related U.S. Application Data
(60) Provisional application No. 63/495,643, filed on Apr. 12, 2023.

(51) Int. Cl. A61B 5/00; A61B 5/252; A61B 5/332; A61B 5/339; A61L 2/10; G16H 20/00; G16H 40/67

(52) U.S. Cl. CPC A61B 5/0006; A61B 5/252; A61B 5/332; A61B 5/339; A61L 2/10; G16H 20/00; G16H 40/67; A61L 2202/122

(57) ABSTRACT

A wireless Electrocardiogram (ECG) device for wirelessly capturing heart activity of a patient. The device includes wireless suction electrodes for placement on the skin of the patient, the electrodes are configured to wirelessly transfer the recorded ECG activity in real time to the device, a monitor of the device displays the ECG data, and a wireless transceiver of the devices enables the device to transmit the ECG data to one or more remote devices. The device includes an amplifier for amplification and noise reduction, a signal processing unit to transform analog signals into digital data, a software to interpret data and provide recommendations. The device includes a drawer for storing the electrodes and includes UV disinfecting light for sanitizing the electrodes.

### CROSS-REFERENCE TO RELATED APPLICATION

The present application claims priority to, and the benefit of, U.S. Provisional Application No. 63/495,643, which was filed on Apr. 12, 2023, and is incorporated herein by reference in its entirety.

### FIELD OF THE INVENTION

The present invention relates generally to the field of electrocardiogram devices. More specifically, the present invention relates to a novel wireless ECG device and system that does not use wires for detecting heart activity. Wireless electrodes are used for placement on a patient and ECG data is wirelessly received by the ECG device. The ECG device displays the ECG graphs and can wirelessly transmit the data to one or more remote devices such as cloud storage, hospital medical records, and more. The device prevents wire tangling, enabling medical professionals to quickly use the ECG. Accordingly, the present disclosure makes specific reference thereto. Nonetheless, it is to be appreciated that aspects of the present invention are also equally applicable to other like applications, devices, and methods of manufacture.

### BACKGROUND

By way of background, Electrocardiograms (ECGs) are used in medical diagnostics to monitor and record the electrical activity of the heart. More specifically, ECGs provide valuable information about the heart's health, rhythm, and potential abnormalities. The information is used by medical professionals to identify various heart conditions, such as arrhythmias, heart attacks, and other cardiac disorders. Commonly, conventional ECG machines are placed inside a healthcare facility for patients as traditional ECG machines, while highly effective, are often large and stationary. Further, traditional ECG machines use multiple wires and leads that are placed on the patient's body. The wires transmit the electrical signals from the body to the machine for analysis. However, the presence of numerous wires can lead to tangling, which not only disrupts the monitoring process but also poses a risk of delaying patient treatment, potentially leading to serious consequences.

It is common for recording ECG of critical patients in ground ambulances, air ambulances, and more for providing effective first aid. However, traditional ECG machines lack portability and thus their use is restricted in scenarios where mobility and immediate monitoring are crucial. Further, the wires connecting the patient to the ECG machine can become disconnected, lost, or broken during movement or transportation. This can result in a loss of critical data and obstruct a medical-personnel from accurately reading the patient's heart rhythm. People desire a portable ECG device that eliminates the need for multiple wires and helps in ECG monitoring in a variety of settings.

Therefore, there exists a long felt need in the art for a portable ECG device that eliminates the need for multiple wires for recording heart activities of a patient. There is also a long felt need in the art for a portable ECG device that can be used for ECG monitoring in a variety of settings, including ground ambulances, air ambulances, and other emergency situations. Additionally, there is a long felt need in the art for a portable ECG device that uses wireless technology for monitoring and transmitting ECG records of a patient. Moreover, there is a long felt need in the art for a wireless ECG system that enables doctors to receive and review an ECG of a patient remotely. Further, there is a long felt need in the art for a wireless ECG device that uses reusable wireless electrodes for placement on a patient's body. Finally, there is a long felt need in the art for a wireless ECG device that eliminates risk of wire disconnection, loss, or breakage, therefore, ensuring continuous and accurate ECG monitoring.

The subject matter disclosed and claimed herein, in one embodiment thereof, comprises a wireless electrocardiogram (ECG) device for capturing and recording a patient's electrocardiogram data. The device comprising a slidable drawer housing a plurality of wireless suction cup electrodes for placement on a human body for recording and wirelessly transmitting heart activity, a UV disinfecting light integrated within the drawer, the UV light is configured to disinfect the plurality of suction cup electrodes when stored therein, and a touch-based display screen for displaying ECG waveforms, patient data, and menu options. The device is wirelessly coupled to external devices such as hospital medical system, a cloud storage, and a printer. The device does not use any wires and therefore, can be used in ambulances and in other constrained space areas.

In this manner, the wireless ECG device of the present invention accomplishes all of the forgoing objectives and provides users with a wireless ECG system capable of offering reliable heart rhythm readings without the need for wires. The device prevents wire tangling, enabling medical professionals to quickly use the ECG rather than spend time worrying about wires. The data can be sent to a remote device, thus, allowing quick inspection by remote doctors for effective and immediate treatment.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a wireless electrocardiogram (ECG) device for capturing and recording a patient's electrocardiogram data. The device comprising a slidable drawer or housing having a plurality of suction cup electrodes for placement on a human body, a UV disinfecting light integrated within the drawer or housing, the UV light is configured to disinfect the plurality of suction cup electrodes when stored therein, and a touch-based display screen for displaying ECG waveforms, patient data, and menu options.

In yet another embodiment, a wireless electrocardiogram (ECG) device for capturing and recording a patient's electrocardiogram data is disclosed. The device includes a drawer or housing incorporating a UV (Ultraviolet) disinfecting light for disinfecting a plurality of suction cup electrodes stored therein, a display screen for providing visualization of ECG waveforms, patient data, and customizable options, the display screen is touch-based and utilizes display technology selected from at least LED, LCD, OLED, and more. The display screen provides a plurality of options including a power button, a fax button, a view button, a contact hospital button, an ECG info button, and a keypad.

In another aspect of the present invention, a wireless electrocardiogram (ECG) system for wirelessly recording ECG of a patient and wirelessly transmitting same to remote sources is disclosed. The system comprising a wireless ECG device equipped with a monitor for displaying real-time ECG recordings, the wireless ECG device comprising suction electrodes for monitoring and recording ECGs, a wireless communication channel enabling connectivity between the wireless ECG device and external devices, the wireless ECG device is capable of transmitting ECG data wirelessly, wherein the external devices can be one of an encrypted cloud storage, a hospital medical records system, and a printer.

A portable ECG device for detecting ECG of a patient without any wires is disclosed. The device includes a plurality of W/10 suction cup electrodes configured for placement on a patient's body, the electrodes are integrated with low power near-field communication (NFC) technology to transmit detected ECG recordings to the ECG device, an amplifier to amplify ECG signals, a signal processing unit for transforming analog amplified signals received from the electrodes into digital signals, and a software for interpreting ECG data to identify heart rate, irregularities, and providing diagnostic recommendations for immediate first-aid.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a schematic view of one potential embodiment of the wireless ECG system of the present invention in accordance with the disclosed architecture;

FIG. 2 illustrates a perspective view of the wireless ECG device of the present invention in accordance with the disclosed structure;

FIG. 3 illustrates a block diagram view showing the electronic components included in the wireless ECG device of the present invention in accordance with the disclosed architecture;

FIG. 4 illustrates a flow chart depicting a process of pairing the electrodes and the wireless ECG device in accordance with the disclosed architecture;

FIG. 5 illustrates a flow chart depicting a process of using the wireless ECG device of the present invention for monitoring ECG of a patient in accordance with the disclosed structure;

FIG. 6 illustrates a flow chart depicting a process of activation and deactivation of the UV light in the wireless ECG device of the present invention in accordance with the disclosed architecture;

FIG. 7 illustrates an enlarged view of the drawer/housing and storage space wherein the drawer/housing is placed inside the wireless ECG device of the present invention in accordance with the disclosed structure; and

FIG. 8 illustrates an isolated view of an exemplary electrode used in the present invention for detecting ECG of a patient in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a portable ECG device that eliminates the need for multiple wires for recording heart activities of a patient. There is also a long felt need in the art for a portable ECG device that can be used for ECG monitoring in a variety of settings, including ground ambulances, air ambulances, and other emergency situations. Additionally, there is a long felt need in the art for a portable ECG device that uses wireless technology for monitoring and transmitting ECG records of a patient. Moreover, there is a long felt need in the art for a wireless ECG system that enables doctors to receive and review ECG of a patient remotely. Further, there is a long felt need in the art for a wireless ECG device that uses reusable wireless electrodes for placement on a patient's body. Finally, there is a long felt need in the art for a wireless ECG device that eliminates risk of wire disconnection, loss, or breakage, thereby ensuring continuous and accurate ECG monitoring.

The present invention, in one exemplary embodiment, is a wireless electrocardiogram (ECG) system for wirelessly recording ECG of a patient and wirelessly transmitting the ECG to remote sources. The system comprising a wireless ECG device equipped with a monitor for displaying real-time ECG recordings, the wireless ECG device comprising wireless suction electrodes for monitoring and recording ECGs, a wireless communication channel enabling connectivity between the wireless ECG device and external devices, the wireless ECG device is capable of transmitting ECG data wirelessly, wherein the external devices can be one of an encrypted cloud storage, a hospital medical records system, and a printer. The wireless ECG device receives heart activity recording from suction electrodes via a NFC channel.

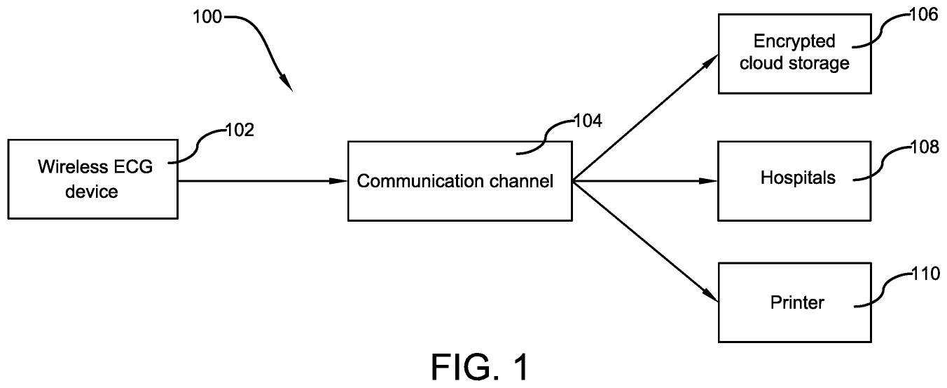

Referring initially to the drawings, FIG. 1 illustrates a schematic view of one potential embodiment of the wireless ECG system of the present invention in accordance with the disclosed architecture. The wireless ECG system 100 is designed to record electrocardiograms (ECGs) of a patient using wireless suction electrodes and transmit the ECG wirelessly to remote devices for further analysis and inspection, while displaying the ECG recordings in real time on a monitor for quick and effective diagnosis and treatment. Advantageously, the wireless ECG detection and transmission system 100 eliminates traditional ECG wires that connect the patient's body to a conventional ECG machine. As a result, the system 100 is useful for ambulances and small places, and helps to prevent delays in patient treatment.

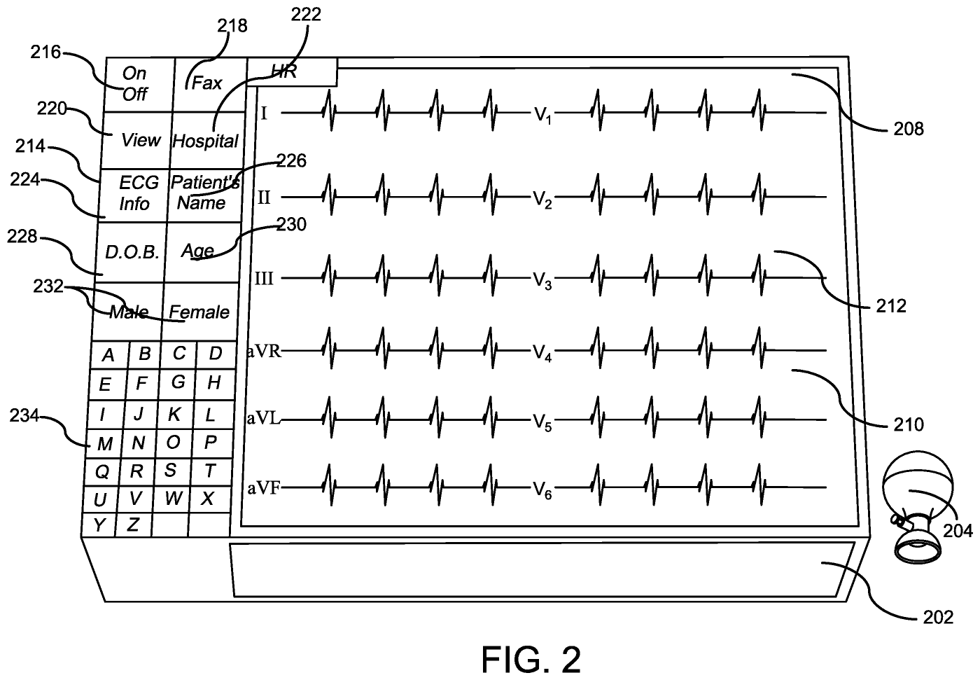

More specifically, the system 100 includes a wireless ECG device 102 which includes a monitor for displaying the ECG recording as illustrated in FIG. 2. The wireless ECG device 102 does not contain any external wire and eliminates tangling and pulling of wires on patient's skin during treatment and ECG recording. The ECG device 102 is a portable device that includes suction electrodes for monitoring recordings and providing insights to a medical professional in real time. The wireless ECG device 102 enables patients and medical professionals greater freedom of movement during ECG recording without the restrictions posed by wired connections.

The ECG device 102 can be connected to a plurality of third-party devices/storages for transmitting ECG information in real time for analysis. As illustrated, the wireless ECG device 102 is connected to a wireless communication channel 104 to connect to at least one of an encrypted cloud storage 106 for storage, a hospital medical record system 108, and/or a printer 110. The encrypted cloud storage 106 can be a central or distributed storage and enables only authorized individuals to access and decipher the stored data. The ECG data stored in the cloud storage 106 can be used for future retrieval and can also be used by regulatory authorities for an investigation.

The wireless ECG device 102 connection to the hospital medical record system 108 enables the device 102 to directly transmit the ECG data for real-time monitoring and analysis by healthcare professionals. It should be noted that device 102 can be connected to one or more hospital medical record systems and a hospital can be manually or automatically selected for the wireless transmission. For physical copies of the ECG recordings, the device 102 is connected to one or more printers 110. This enables for the generation of physical copies of ECG reports easily for inspection. The wireless ECG system 100 of the present invention is designed to transfer ECG data faster than traditional wired systems and has less potential for breakage or signal loss.

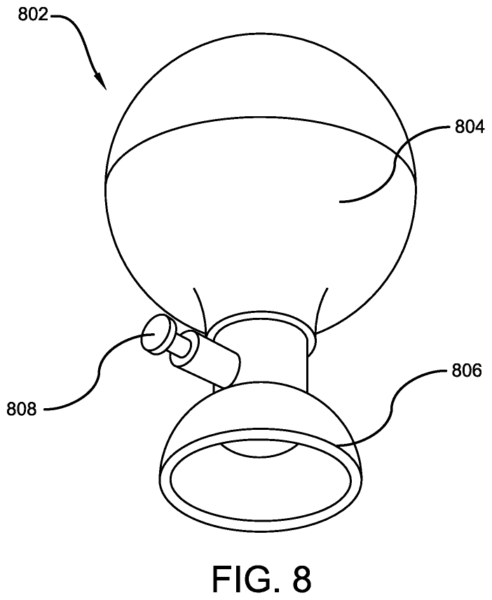

FIG. 2 illustrates a perspective view of the wireless ECG device of the present invention in accordance with the disclosed structure. The ECG device 102 captures and records a patient's electrocardiogram (ECG) data, which represents the electrical activity of the heart. The device 102 features a drawer or housing 202 that houses a plurality (preferably 10) of suction cup electrodes 204 for placement on a human body. The electrodes 204 provide a 12-lead ECG setup, where six chest electrodes and four limb electrodes are used in a specific arrangement to provide comprehensive heart activity information of a patient. Details of each electrode is described in detail in FIG. 8.

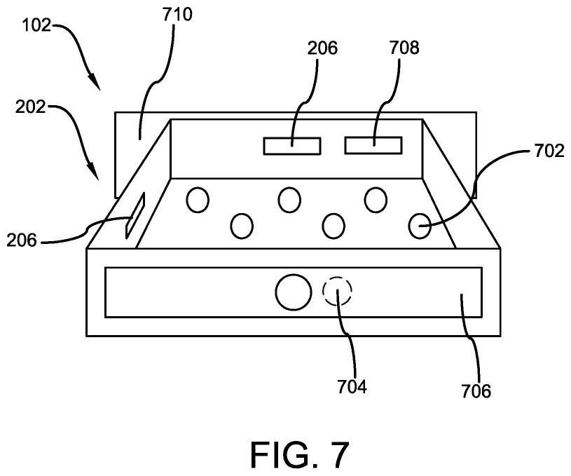

The drawer or housing 202 is equipped with a UV (Ultraviolet) disinfecting light 206 (shown in FIG. 7). The UV light 206 kills or inactivates a variety of microorganisms, including bacteria and viruses and helps in disinfecting the electrodes 204 when stored therein. The UV light 206 makes the electrodes sanitized and reusable.

The device 102 has a display screen 208 that shows various information, such as ECG waveforms, patient data, and menu options. A portion 210 of the display screen 208 displays the ECG waveform and associated parameters 212 and a second portion 214 displays the customization and information options. The display screen 208 is touch-based and can use any display technology such as LED, LCD, OLED, and more.

A power button 216 is configured to turn the device 102 on or off. The power button 216 enables a user to control the overall operation and power status of the device 102. A fax button 218 enables the device 102 to send ECG reports to hospital systems 106 via fax protocol for analysis.

A view button 220 enables users to access ECG recordings stored in the device 102 for review and analysis. In one embodiment, a history of previous ECG tests recorded in the device 102 are selected and displayed using the view button 220. A contact hospital button 222 sends the digital copy of the ECG record to a designated hospital or healthcare facility. The device 102 can also initiate a call or message to alert medical personnel about the patient's condition or to seek assistance.

An ECG info button 224 enables a user to configure the device 102 for recording ECG. The measurement parameters, measurement threshold, and more can be configured using the ECG info button 224. A plurality of buttons such as name button 226, date of birth button 228, age button 230, and gender button 232 are used to input essential patient information before or during the ECG procedure. The input data can be used by the device 102 for providing insights and analysis based on the recorded ECG and provides the patient data as metadata for storage of the ECG. A keypad 234 to input alpha-numeric data in the device 102 is provided in the portion 214 and can display the data in multiple languages allowing the users to work in different languages.

It should be appreciated that the device 102 not only provides a sanitized space for storage of the electrodes used for recording ECG but wirelessly transmits the ECG data to remote devices and displays on the monitor. Further, a plurality of options is provided for a user to configure the device 102 for accurate ECG recording.

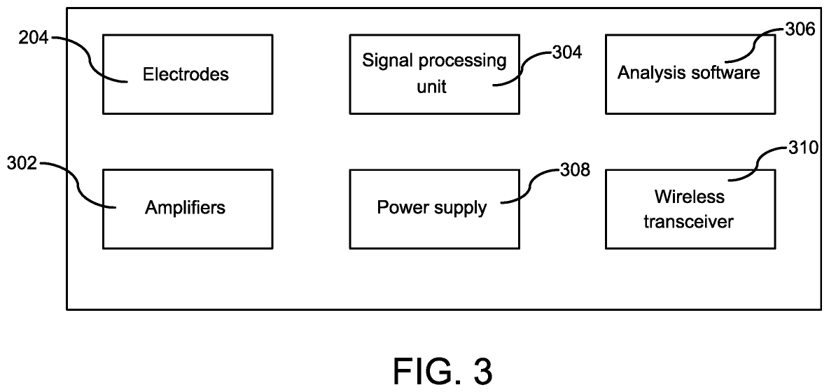

FIG. 3 illustrates a block diagram view showing the electronic components included in the wireless ECG device 102 of the present invention in accordance with the disclosed architecture. W/10 suction cup electrodes 204 are included in the device 102 for placement on a patient's body. The electrodes 204 are integrated with low power near-field communication (NFC) for transmitting the detected recordings to the device 102. The electrodes 204 preferably are used for “12-lead ECG” configuration for providing recording of a comprehensive heart's electrical activity from various angles.

The signals received from the electrodes 204 are weak and therefore, are amplified by an amplifier 302 included in the device 102. The amplifier 302 minimizes noise interference and improves strength of the ECG signals for accurate measurement. Preferably, the amplifier 302 can be an operation amplifier and include common-mode rejection ratio (CMRR). The amplified signals are processed by a signal processing unit 304 which is configured to transform the analog amplified signals into digital signals for representation on the monitor. The digital signals are stored inside the device 102 as well as are transmitted to the remote devices.

The signal processing unit 304 works with analysis software 306 which is used for interpreting the ECG data to identify heart rate, irregularities, and provides diagnosis recommendation for immediate first-aid. The software 306 can be machine-readable instructions and can be updated with new algorithms using over-the-air (OTA) updates. For providing electric power to the device 102, a power supply 308 in the form of one or more replaceable and rechargeable batteries is integrated in the device 102. Preferably, the power supply 308 is disposed in the housing 202 and can be AA or AAA battery.

A wireless transceiver 310 is used for establishing the communication channel to transmit ECG data and other information to remote devices as described in FIG. 1. The transceiver 310 also provides an NFC communication with the electrodes 204 for receiving recordings from the electrodes. The signal processing unit 304 and the software 306 may implement machine learning and artificial intelligence for interpreting ECG recordings and provide insights. Also, the software 306 can perform predictive analytics and may recommend medicines for instant relief to a patient based on the ECG recordings and profile of the patient.

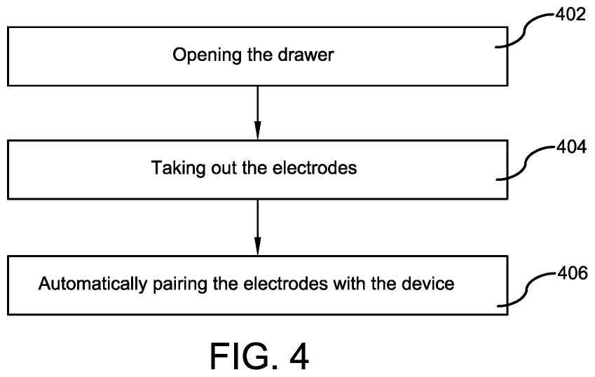

FIG. 4 illustrates a flow chart depicting a process of pairing the electrodes and the wireless ECG device in accordance with the disclosed architecture. Initially, the drawer 202 which stores electrodes in the device 102 is opened by a user for accessing the electrodes for placement on a patient (Step 402). It should be noted that the UV disinfecting light is automatically turned off when the drawer is opened to access the electrodes 204. Then, the user takes out the electrodes from the drawer 202 (Step 404) and the electrodes 204 are wireless and are compact in size and shape. As soon as the electrodes are taken out, the electrodes 204 are automatically paired with the device 102 using NFC for allowing the electrodes 204 to transmit the recordings in real time for analysis and display on monitor of the device 102 (Step 406).

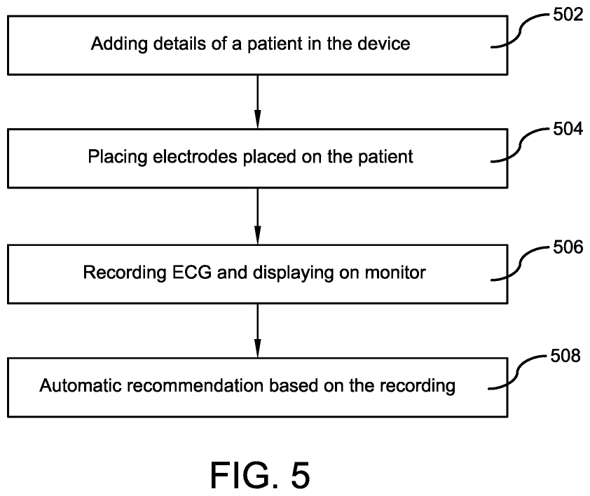

FIG. 5 illustrates a flow chart depicting a process of using the wireless ECG device 102 of the present invention for monitoring ECG of a patient in accordance with the disclosed structure. Initially, details such as age, name, and gender of a patient are added to the device 102 using the name button 226, date of birth button 228, age button 230, and gender button 232 (Step 502). Then, the electrodes 204 are placed on the patient for detecting ECG details (Step 504). Thereafter, the electrodes 204 record the ECG data and wirelessly transmits to the device 102 using the NFC or any other wireless communication protocol (Step 506). The ECG data is processed, analyzed, and displayed as described in FIGS. 2 and 3. Finally, the software system 306 using the profile of the patient and recordings, provides an automatic recommendation citing at least medication and other first aid (Step 508).

In some embodiments of the present invention, the device 102 may recommend and display nearby hospitals and health care facilities depending on the current location of the device 102.

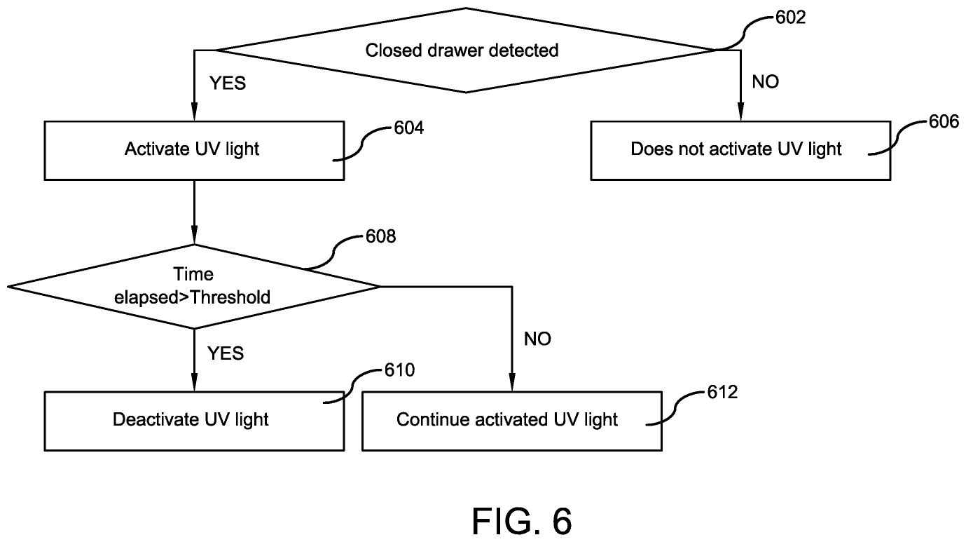

FIG. 6 illustrates a flow chart depicting a process of activation and deactivation of the UV light in the wireless ECG device of the present invention in accordance with the disclosed architecture. Initially, it is determined if housing 202 is closed or not and can be detected by a sensor as illustrated in FIG. 7 (Step 602). In case the drawer 202 is determined to be closed, then, the UV light is automatically activated (Step 604) and in case the drawer 202 is open, then UV light is not activated by the device 102 (Step 606).

When the UV light is activated, then the time elapsed since the activation of the UV light is determined and a comparison with a threshold value (such as 2 minutes, 5 minutes, and the like) is performed (Step 608). In case the time elapsed is more than the threshold value, then the UV light is automatically deactivated (Step 610), otherwise the UV light is activated until the time lapsed is equal to the threshold value (Step 612).

FIG. 7 illustrates an enlarged view of the housing and storage space in which the drawer is placed inside the wireless ECG device 102 of the present invention in accordance with the disclosed structure. The drawer or housing 202 is slidably positioned in a storage space 710 of the device 102 and includes a plurality of spaces 702 for accommodating electrodes 204 and has the UV light 206 positioned along one or more walls of the drawer 202. A sensor 704 is disposed at the front wall 706 of the drawer or housing 202 and is configured to detect open or closed status of the drawer 202. A timer module 708 is configured to detect the time lapsed from closing of the drawer 202 and can automatically activate or deactivate the UV light 206.

FIG. 8 illustrates an isolated view of an exemplary electrode used in the present invention for detecting ECG of a patient in accordance with the disclosed structure. The electrode 802 includes a rubber bulb 804, a contact surface 806, and an NFC enabled lead wire terminal 808. The NFC lead wire terminal 808 wirelessly transmits the recordings to the ECG device 102 for analysis and display without use of conventional wires used in wired electrodes. The rubber bulb 804 helps create a gentle suction to ensure proper contact between the electrode and the skin of a patient, improving the signal quality for ECG measurements.

In the preferred embodiment, each electrode used in the system 100 has a unique identifier encoded therein which is also transmitted along the ECG data to the device 102 for allowing the device 102 to accurately detect and map the readings with placement of the electrode on the skin of the patient.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “wireless ECG detection and transmission system”, “wireless ECG system”, and “system” are interchangeable and refer to the wireless ECG detection and transmission system 100 of the present invention. Similarly, as used herein “wireless ECG device”, “ECG device”, and “device” are interchangeable and refer to the wireless ECG device 102 of the present invention.

Notwithstanding the forgoing, the wireless ECG detection and transmission system 100 and the wireless ECG device 102 of the present invention can be of any suitable size and configuration as is known in the art without affecting the overall concept of the invention, provided that it accomplishes the above stated objectives. One of ordinary skill in the art will appreciate that the wireless ECG detection and transmission system 100 and the wireless ECG device 102 as shown in the FIGS. 1-2 are for illustrative purposes only, and that many other sizes and shapes of the wireless ECG detection and transmission system 100 and the wireless ECG device 102 are well within the scope of the present disclosure. Although the dimensions of the wireless ECG detection and transmission system 100 and the wireless ECG device 102 are important design parameters for user convenience, the wireless ECG detection and transmission system 100 and the wireless ECG device 102 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A wireless ECG system for recording electrocardiograms (ECGs) of a patient, the wireless ECG system comprising:
a wireless portable ECG device having a plurality of wireless electrodes, a monitor for displaying an ECG recording, and a wireless communication channel for transmitting said ECG recording to a remote device;
wherein said monitor accessible for further analysis and inspection at said remote device; and
further wherein said displaying said ECG recording is real time.

2. The wireless ECG system of claim 1, wherein said wireless communication channel is connected to the group consisting of at least one of an encrypted cloud storage, a hospital medical record system, and a printer.

3. The wireless ECG system of claim 2, wherein said plurality of wireless electrodes are wireless suction electrodes.

4. The wireless ECG system of claim 1, wherein said wireless communication channel is connected to a plurality of third-party devices.

5. The wireless ECG system of claim 1, wherein said wireless communication channel is connected to an encrypted cloud storage, and further wherein said encrypted cloud storage is selected from the group consisting of a central storage and a distributed storage.

6. The wireless ECG system of claim 5, wherein said encrypted cloud storage accessible only by authorized individuals.

7. The wireless ECG system of claim 1, wherein said wireless communication channel is connected to a hospital medical record system for directly transmitting said ECG recording for said real time monitoring and analysis by healthcare professionals.

8. The wireless ECG system of claim 1, wherein said wireless communication channel is connected to a plurality of hospital medical record systems for directly transmitting said ECG recording for said real time monitoring and analysis by healthcare professionals.

9. The wireless ECG system of claim 1, wherein said wireless communication channel is connected to one or more printers for generation of physical copies of said ECG recordings.

10. A wireless ECG system for recording electrocardiograms (ECGs) of a patient, the wireless ECG system comprising:
a wireless portable ECG device having a plurality of wireless electrodes, a monitor for displaying an ECG recording, a wireless communication channel for transmitting said ECG recording to a remote device, and a housing;
wherein said monitor accessible for further analysis and inspection at said remote device; and
further wherein said housing having a drawer for housing said plurality of wireless electrodes and a UV light for disinfecting said plurality of wireless electrodes stored in said drawer.

11. The wireless ECG system of claim 10, wherein said displaying said ECG recording is real time.

12. The wireless ECG system of claim 10, wherein said wireless communication channel is connected to the group consisting of at least one of an encrypted cloud storage, a hospital medical record system, and a printer.

13. The wireless ECG system of claim 10, wherein said plurality of wireless electrodes are wireless suction electrodes.

14. The wireless ECG system of claim 10, wherein said wireless communication channel is connected to an encrypted cloud storage, and further wherein said encrypted cloud storage is selected from the group consisting of a central storage and a distributed storage.

15. The wireless ECG system of claim 10, wherein said wireless communication channel is connected to at least one hospital medical record system for directly transmitting said ECG recording for said real time monitoring and analysis by healthcare professionals.

16. The wireless ECG system of claim 10, wherein said wireless communication channel is connected to one or more printers for generation of physical copies of said ECG recordings.

17. A wireless ECG system for recording electrocardiograms (ECGs) of a patient, the wireless ECG system comprising:
a wireless portable ECG device having a plurality of wireless electrodes, a monitor for displaying an ECG recording, a wireless communication channel for transmitting said ECG recording to a remote device, and a housing;
wherein said plurality of wireless electrodes integrated with a low power near-field communication (NFC) for transmitting said ECG recording to said remote device;
wherein said NFC amplified by an amplifier for increasing strength of the transmitting of said ECG recording;
wherein said monitor accessible for further analysis and inspection at said remote device; and
further wherein said housing having a drawer for housing said plurality of wireless electrodes and a UV light for disinfecting said plurality of wireless electrodes stored in said drawer.

18. The wireless ECG system of claim 17, wherein said displaying said ECG recording is real time.

19. The wireless ECG system of claim 17, wherein said wireless communication channel is connected to the group consisting of at least one of an encrypted cloud storage, a hospital medical record system, and a printer.

20. The wireless ECG system of claim 17, wherein said plurality of wireless electrodes are wireless suction electrodes.
